(19) United States
(12) Patent Application Publication
(10) Pub. No.: US 2010/0047830 A1
(43) Pub. Date: Feb. 25, 2010

(54) COMPOSITIONS AND METHODS FOR DETECTING CANCERS IN A SUBJECT

(75) Inventors: Joseph Katz, Gainesville, FL (US); Edward Chan, Gainesville, FL (US)

Correspondence Address: Gregory A. Nelson;Novak Druce & Quigg LLP, 525 Okeechobee Blvd, Suite 1500, West Palm Beach, FL, 33401, US

(73) Assignee: University of Florida Research Foundation, Inc., Gainesville, FL (US)

(21) Appl. No.: 12/523,271

(22) PCT Filed: Jan. 31, 2008
(86) PCT No.: PCT/US08/52673

§ 371 Date: Jul. 15, 2009

Related U.S. Application Data
(60) Provisional application No. 60/887,659, filed on Feb. 1, 2007.

(51) Int. Cl. G01N 33/53; C07K 16/18

(52) U.S. Cl. 435/7.92; 530/389.1; 435/7.1

(57) ABSTRACT

Disclosed are compositions and methods for detecting oral and gastrointestinal cancers in a subject. Autoantigen p90 was shown to be overexpressed in oral cancer cells, and this protein, as well as its companion autoantigen p62 and antibodies directed to both proteins, can be used as markers for detecting oral digestive and other cancers in a subject at an early stage.

### BACKGROUND OF THE INVENTION

Oral digestive cancers (e.g., oral cancer, colorectal cancer (CRC), gastrointestinal (GI) cancer) are among the most common cancers suffered by Americans, and CRC is the number three cancer killer of both men and women in the United States. In excess of 40 billion dollars of health-care expenditures are used for gastrointestinal diseases annually, more than half on cancer. CRC is often diagnosed at a late stage with concomitant poor prognosis. Early detection greatly improves prognosis; however, the invasive, unpleasant and inconvenient nature of current diagnostic procedures limits their applicability. Procedures for diagnosis such as endoscopy and colonoscopy are complicated, expensive, and not desired by patients. No serum-based test is currently of sufficient sensitivity or specificity for widespread use. Detecting oral digestive cancer at an early stage is particularly problematic because there are no clinical symptoms present at the initial stages, and there are no serological markers for early detection. When diagnosis is established at late stages, the prognosis is ominous.

### SUMMARY OF THE INVENTION

The invention relates to the discovery that autoantigen p90 (also referred to herein as CIP2A) is overexpressed in oral digestive cancer cells. This protein, its companion autoantigen p62, and antibodies directed to both proteins can be used as markers for detecting oral digestive and other cancers in a subject at an early stage. Based on the foregoing, an enzyme-linked immunosorbent assay (ELISA) capture assay was developed which can reliably analyze p90 (a protein associated with some solid tumors) expression in a sample and be used to detect cancer (e.g., oral cancer, colon cancer, and other oral-digestive cancer and other non-solid cancer) in a serum, saliva or blood sample, at concentrations as low as 1 nanogram (see Table 1). This assay was used to examine the serum of 22 human cancer patients and 7 controls. The ELISA test was able to distinguish normal serum from cancer patients with a sensitivity of 76% and specificity of 86%. When looking at a specific group of nonsolid malignant tumors comprised of 10 patients and 7 controls, the sensitivity increased to 86% and the specificity remained 86%. After establishing a standard curve using decreasing concentration of recombinant p-90, human serum was tested. The invention as described herein includes ELISA assays analyzing the expression of p90, ELISA assays analyzing the expression of p62, and ELISA assays analyzing the expression of both p90 and p62.

Accordingly, the invention features a composition for detecting the presence of cancer cells in a biological sample. The composition includes an agent that specifically binds p62 or p90, such as an antibody. The cancer cells can be oral cancer cells, GI cancer cells, and CRC cells as examples.

In another aspect, the invention features a method of detecting oral or gastrointestinal cancer cells in a subject. The method includes the steps of: (a) providing a biological sample from the subject; (b) detecting the amount of p62 and/or p90 in the sample; and (c) correlating the amount of the p62 or p90 in the sample with the presence of oral or gastrointestinal cancer in the subject. The step (b) of detecting the amount of p62 or p90 in the sample can include contacting the sample with at least one antibody that specifically binds the p62 or the p90, including performing an ELISA. The biological sample can be a fluid such as saliva, blood, plasma, or serum. The cancer cells can be oral cancer cells, GI cancer cells, and CRC cells as examples. In a typical method, elevated levels of p62 and/or p90 indicate the presence of cancerous cells.

In still another aspect, the invention features a kit for detecting oral or gastrointestinal cancer cells in a subject. The kit includes (a) a solid substrate; (b) at least one antibody that binds specifically to p62 or p90; (c) an agent for detecting binding of the at least one antibody to the p62 or p90; and (d) instructions for using the kit to detect oral or gastrointestinal cancer cells in a subject. The at least one antibody is typically a monoclonal antibody. The agent for detecting binding of the at least one antibody to P90 can include a chromogenic substrate molecule. Detecting binding of the at least one antibody to P90 is correlated with cancer.

Unless otherwise defined, all technical terms used herein have the same meaning as commonly understood by one of ordinary skill in the art to which this invention belongs.

By the terms “p62”, “p62 marker”, “p62 protein” or “p62 polypeptide” are meant an expression product of a p62 gene such as a native p62 protein, or a protein that shares at least 65% (but preferably 75, 80, 85, 90, 95, 96, 97, 98, or 99%) amino acid sequence identity with one of the foregoing and displays a functional activity of a mammalian native p62 protein. Similarly, by the terms “p90”, “p90 marker”, “p90 protein” or “p90 polypeptide” are meant an expression product of a p90 gene such as a native p90 protein, or a protein that shares at least 65% (but preferably 75, 80, 85, 90, 95, 96, 97, 98, or 99%) amino acid sequence identity with one of the foregoing and displays a functional activity of a mammalian native p90 protein. A “functional activity” of a protein is any activity associated with the physiological function of the protein. Accession numbers AF057352 and NM_{—}006548 disclose human p62 protein amino acid sequences and corresponding nucleic acid sequences. Accession numbers AF334474 and NM_{—}006548 disclose human p90 protein amino acid sequences and corresponding nucleic acid sequences.

As used herein, the terms “p62-specific antibody” and “antibody to p62” mean an antibody that binds p62 and displays no substantial binding to other naturally occurring proteins other than those sharing the same antigenic determinants as p62. The terms include polyclonal and monoclonal antibodies.

By the terms “p90-specific antibody” and “antibody to p90” are meant an antibody that binds p90 and displays no substantial binding to other naturally occurring proteins other than those sharing the same antigenic determinants as p90. The terms include polyclonal and monoclonal antibodies.

As used herein, “bind,” or “binds,” means that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ liters/mole for that second molecule.

The term “subject,” as used herein, means a human or non-human animal, including but not limited to mammals such as a dog, cat, horse, cow, pig, rabbit, guinea pig, sheep, goat, primate, rat, and mouse.

The term “serum,” as used herein, means the fluid that separates from the clot in the coagulation of blood.

By reference to an “antibody that specifically binds” to another molecule is meant an antibody that binds the other molecule, and displays no substantial binding to other naturally occurring proteins other than those sharing the same antigenic determinants as the other molecule. The term “antibody” includes polyclonal and monoclonal antibodies as well as antibody fragments or portions of immunoglobulin molecules that can specifically bind the same antigen as the intact antibody molecule.

Although compositions, methods and materials similar or equivalent to those described herein can be used in the practice or testing of the present invention, suitable compositions, methods and materials are described below. All publications, patent applications, patents and other references mentioned herein are incorporated by reference in their entirety. In the case of conflict, the present specification, including definitions will control. The particular embodiments discussed below are illustrative only and not intended to be limiting.

### BRIEF DESCRIPTION OF THE DRAWINGS

There is shown in the drawings embodiments which are presently preferred, it being understood, however, that the invention can be embodied in other forms without departing from the spirit or essential attributes thereof.

FIG. 1 is a pair of photographs showing serial sections of a human oral cancer stained with rabbit anti-p90 (left panel) with the nuclei counterstained using DAPI. The over-expression of p90 was primarily observed in the cytoplasm of the cancer cells. The section stained with control preimmune serum from the same rabbit shows little or no staining (right panel).

FIG. 2 is a photograph of an ELISA setup plate from standard dilution of recombinant p90.

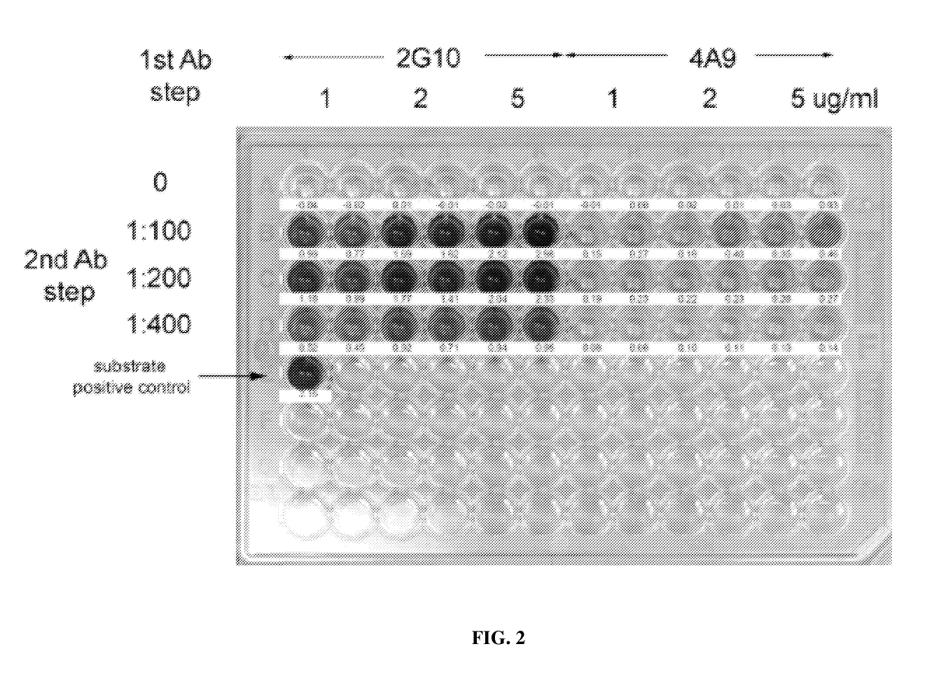

FIG. 3 is a pair of photographs of a control sample immunostained with preimmune serum (left panel) and a sample immunostained with polyclonal rabbit anti-CIP2A serum (right panel).

FIG. 4 is a pair of photographs of oral squamous cell carcinoma in situ tissue positive for p90 expression (right panel) and a negative control [OF WHAT??] (left panel).

FIG. 5 is a table of an ELISA plate setup for patients serum and controls.

### DETAILED DESCRIPTION OF THE INVENTION

The invention relates to compositions and methods for detecting oral digestive and other cancers in a subject (e.g., humans). The below described preferred embodiments illustrate adaptations of these methods and compositions. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

### Compositions for Detecting Oral Digestive and Other Cancers

The invention provides compositions for detecting oral digestive and other cancers in a subject. An example of such a composition is one including an agent that specifically binds p62 or p90. Typically, the agent that specifically binds p62 or p90 is an antibody. Antibodies to p62 and p90 have been prepared by methods well known to those of skill in the art to provide antibody compositions. Monoclonal antibodies, polyclonal antibodies, and antibody derivatives are useful in compositions and methods described herein. Any antibody that binds to p62 or p90 is suitable for use in the invention.

### Detecting Oral and Gastrointestinal Cancer Cells

The invention provides a method for detecting oral digestive and other cancer cells in a subject. The method includes the steps of providing a biological sample from the subject, detecting the amount of p62 and/or p90 in the sample, and correlating the amount of the p62 or p90 in the sample with the presence of oral digestive or other cancer in the subject. In a typical method, an elevated level of p62 and/or p90 in the sample indicates the presence of cancer. In methods described herein, subjects are mammals including rodents and humans. A biological sample can be blood, serum, plasma or saliva, as well as esophageal, gastric, colon, or hepatic tissue, or any other suitable tissue for detecting oral digestive or other cancer cells.

Methods for detecting oral digestive and other cancer cells involve providing a biological sample from the subject and contacting the biological sample with a reagent (e.g., an antibody) that binds p62 or p90 for detecting the amount of p62 and/or p90 in the sample. Typically, the biological sample is contacted with an antibody that specifically binds p62 or p90 to examine the biological sample for overexpression of the protein. As described above, the antibody may be a monoclonal or polyclonal antibody, as well as an antibody fragment. Detecting the amount of p62 or p90 in the sample typically includes performing an ELISA. Any suitable methods, however, for detecting the amount of p62 or p90 in a biological sample can be used. For example, multiplex assays including protein microarrays and Luminex-based systems can be used.

### Kits

The invention includes a kit for assaying the levels of p90, p62, or both p90 and p62 in a biological sample such as saliva, plasma, or serum (e.g., to detect the presence of cancer in a subject). In one embodiment, a kit includes a solid substrate, at least one capture antibody that binds specifically to p90, another antibody specific for the antibody used to detect the p90 bound to the capture antibody, and instructions for using the kit to analyze p90 expression in a sample from a subject and detect the presence of a cancer in the subject. The kit typically includes p90-specific polyclonal, monoclonal or recombinant antibodies immobilized on ELISA plates, glass slides or other suitable substrates. The immobilized antibody is incubated with the biological sample allowing binding of the p90 that may be contained in the sample. The binding of the p90 is determined by a detection antibody specific for p90. The presence of the detection antibody is visualized and quantified by detection agents such as enzyme-linked antibodies reactive with the detection antibody. The presence of the enzyme linked antibody is detected using chromogenic substrate molecules appropriate for the enzyme. Quantitation of the signal can then be performed by optical density measurements at the wavelength optimum for the particular chromagen. More complex approaches utilize surface plasmon resonance, fluorescence resonance energy transfer or other techniques which involve the use of specialized equipment to assay binding may have advantages in terms of quantifying binding and for high-throughput applications.

### EXAMPLES

### Example 1

### p90 is Overexpressed in Oral Cancer Cells

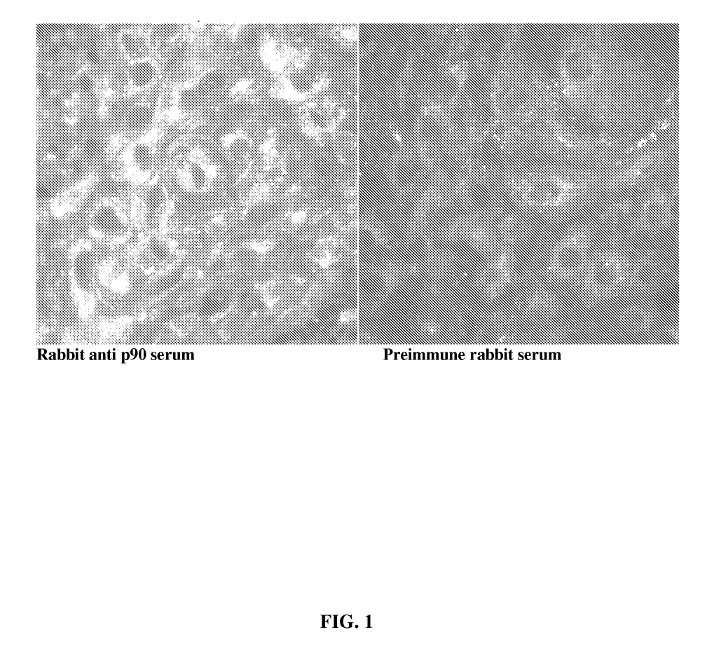

Referring to FIG. 1, serial sections of a human oral cancer were stained with rabbit anti-p90 (left panel) with the nuclei counterstained using DAPI. The over-expression of p90 was primarily observed in the cytoplasm of the cancer cells. The section stained with control preimmune serum from the same rabbit shows little or no staining (right panel). The methods used to show overexpression of p90 in oral cancer cells are described in Soo-Hoo et al., Oncogene 21:5006-5015, 2002.

### Example 2

### Antibodies to Cancer-Associated Autoantigens

The production of autoantibodies in cancer is thought to be due to autoimmune responses directed to cellular proteins that are overexpressed or aberrantly regulated as a result of cellular transformation to cancer. Two cancer-associated autoantigens p62 and p90 were identified (Zhang et al., J. Exp. Med. 189:1101-1110, 1999; Soo-Hoo et al., Oncogene 21:5006-5015, 2002). A rabbit polyclonal antibody as well as two mouse IgG monoclonal antibodies to p90 were developed and are being characterized for use in detecting early cancer cells. Also, two monoclonal antibodies to p62 were developed.

### Example 3

### Evaluating p62 and p90 as Early Cancer Biomarkers

Subsets of oral digestive or other cancers are examined using archival pathology samples and appropriate controls from other tissues. As stated above, p90 is overexpressed in some cancers (e.g., oral cancer, GI cancer) and can be used as a marker for cancer (e.g., oral cancer, GI cancer). To evaluate p62 and p90 as early cancer biomarkers, an immunoassay such as an ELISA is developed for the detection of autoantibodies to these proteins in sera and saliva from one or more patient populations, the patients having cancers of the digestive system. In addition, a sandwich ELISA-type assay is developed to detect the release of p90 (p90 is a “companion” autoantigen of p62) and p62 in the same biological fluids. The polyclonal and monoclonal antibody reagents described in Example 2 are used in these assays to examine the expression of p62 and p90 in cancer cells and to establish these proteins as novel biomarkers for oral digestive or other cancers. An example of a diagnostic test for detecting cancer early in a subject using the antibodies to p62 and p90 is the salivary spit test. This test has several advantages, including: simplicity—the test can be performed anywhere; the test is non-invasive; the test is inexpensive compared to other procedures currently used; it is ideal for large scale screening; and the test provides for a rapid diagnosis.

### Example 4

### Diagnosing Oral Digestive and Other Cancers Early

A study to determine the prevalence of the proteins p90 and p62 in plasma and saliva of subjects with oral digestive cancer is performed. The study is accomplished through the screening of plasma and saliva fluids in subjects that have been just diagnosed with either oral, esophageal, gastric, colon, or hepatic cancer. Results from the study are used to develop a commercial ELISA kit for the early detection of these cancers which can then be used in the offices of physicians (e.g., general practitioners) as well as hospitals and other medical institutions.

For this research, 150 subjects are studied and these subjects are selected based on the type of oral digestive cancer they have. The study can include, for example, six groups with twenty five subjects in each group. The subject groups are as follows: oral cancer, gastric cancer, colon cancer, esophageal cancer, hepatic cancer, and a control group. A collection of both 15 milliliters of saliva and 10 milliliters of blood are obtained from each subject. The samples are analyzed for the presence of the proteins p90 and p62.

In a typical protocol, 10 ml of blood are collected from each subject. 15 ml of whole mouth saliva are also collected from the subjects by the patients spitting into a sterile tube. The blood and saliva are then centrifuged and the supernatant and pellet are collected and submitted for analysis by ELISA using the p90 and p62 antibodies.

### Example 5

### Aberrant Expression of p90 in Oral Squamous Cell Carcinoma

P90 is an autoantibody to a 90 kDa cytoplasmic protein p90 has been identified as an inhibitor of an important cellular protein phosphatase 2A (PP2A) and has been named Cancerous Inhibitor of PP2A (CIP2A). See Junttila, M. R., P. Puustinen, M. Niemela, R. Ahola, H. Arnold, T. Bottzauw, R. Ia-Aho, C. Nielsen, J. Ivaska, Y. Taya, S. L. Lu, S. Lin, E. K. L. Chan, X. J. Wang, R. Grenman, J. Kast, T. Kallunki, R. Sears, V. M. Kahari, and J. Westermarck, CIP2A Inhibits PP2A in Human Malignancies, Cell 130:51-62, 2007. CIP2A is overexpressed in human head and neck squamous cell carcinoma and colon cancer. CIP2A could potentially be a marker for many different types of cancer in various areas of the body. To determine if CIP2A is overexpressed in oral squamous cell carcinomas, samples from a cohort of patients with five year survival data were examined.

### Methods

Parafilm block samples were obtained from 25 patients diagnosed with oral cancer. The samples were retrieved from different areas within the oral cavity, including the tongue and palate, as well as from the larynx, lymph nodes, and tonsils. Four micron sections were cut, antigen retrieval processed, and immunostained with a polyclonal rabbit anti-CIP2A serum. Pre-immune serum and rabbit anti-giantin antibodies were also used as negative and positive controls, respectively. The expression level of the CIP2A antigen was determined by immunofluorescence and scored by two pathologists for intensity on a scale 1-3.

### Results

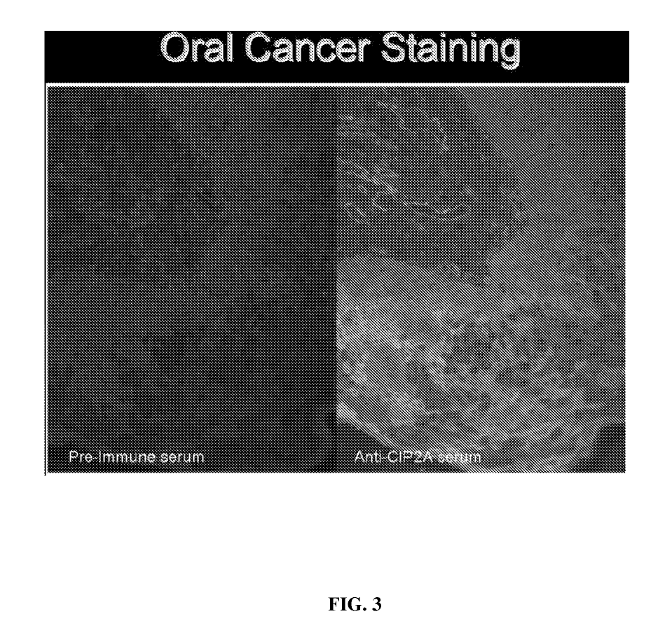

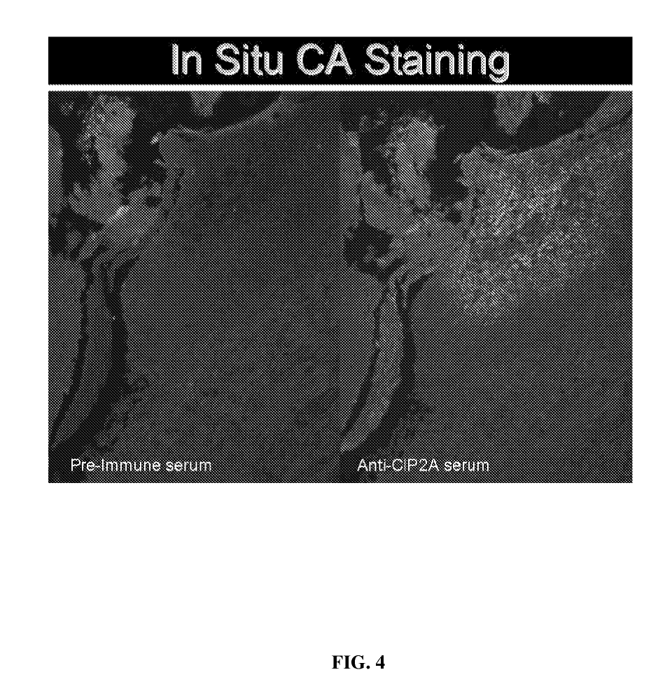

All of the samples were positive with the anti-giantin antibodies indicating that the antigen retrieval method and staining protocols were successful. All 25 showed a greater intensity of the fluorescent signal in the anti-CIP2A than the pre-immune CIP2A serum. See FIGS. 3 and 4. The positive anti-CIP2A samples also contained areas of variable levels of CIP2A expression. The areas that contained tumor showed little aberrant expression (only 25% of these section had intense staining), while the near-adjacent epithelium displaying dysplastic features and occasional foci of in-situ changes exhibited aberrant expression of CIP2A (more than 75%).

### CONCLUSION

CIP2A aberrant expression is present in oral squamous cell carcinoma with the strongest intensities observed in the non-invasive component and better differentiated areas. Further analysis is needed to explain the difference in expression within these particular areas of the tissue samples.

### Example 6

### ELISA Protocol

1. Coating: Use 2G10 (6 mg/ml) in half the plate and use 4A9 (11 mg/ml) in the other half. Use 1 ug/ml in columns 1-2 and 7-8, use 2 ug/ml in columns 3-4 and 9-10, and use 5 ug/ml in columns 5-6 and 11-12. Use 100 ul per well. Cover with saran wrap and incubate overnight at 4° C.

2. Blocking Step. Add 200 ul of post-coating solution. Invert and decant solution. Add 300 ul of post coating solution. Incubate at room temperature for a minimum of 2 hours or place in 4° C. for up to a month.

3. Make antigen solution before wash

4. Decant post coating solution. Wash plate for 3 seconds (3×) with 1×PBS-Tween.

5. Make 100 ng/ml antigen, p90, solution and add 200 ul of solution per well. Incubate at room temperature for 1.5-2 hours on shaker.

6. Before wash, dilute second antibody in serum diluent.

7. Decant antigen solution. Wash plate for 3 seconds (3×) with 1×PBS-Tween.

8. Use Rabbit 5181, in row A 0, in row B 1:100, in row C 1:200 and in row D 1:400. Use 200 ul per well. Incubate at room temperature for 1.5-2 hours on shaker.

9. Make third antibody before wash.

10. Decant secondary antibody. Wash plate for 3 seconds (3×) with 1×PBS-Tween.

11. Use HRP-goat antibody (1:5 K) and dilute in anti-Ig diluent, use 200 ul per well. Incubate at room temperature for 1.5-2 hours on shaker.

12. Make the working substrate solution:

| | | |
|---|---|---|
| a) ABTS | 2 | ml |
| b) Mcllvain's buffer | 18 | ml |
| c) H₂O₂ (1%) | 0.1 | ml |

13. Decant and wash for 2 seconds (2×) in 1×PBS-Tween followed by a wash for 3 seconds (3×) in 1×PBS.

14. Add 200 ul of working solution. Cover and incubate at room temperature on shaker.

15. Read plates at 405 nm after 25 minutes of incubation and then again after an hour of incubation

### Example 7

### ELISA Results

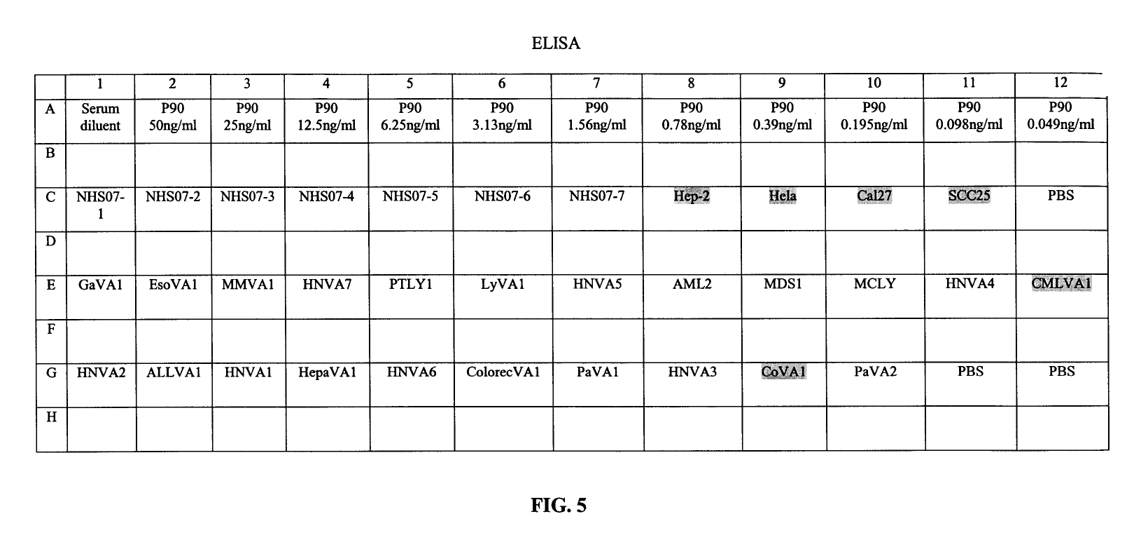

The ELISA protocol described in Example 6 was used to examine p90 expression in human samples. Referring to the table in FIG. 5, an ELISA plate setup for patients serum and controls is shown. In this table, column A is a serial dilution of standard p90; column C is serum from healthy controls (rows 1-7) and cell line lysate controls (rows 8-11); column E is serum from cancer patients (rows 1-12); and column G is serum from cancer patients (rows 1-10) and PBS (rows 11 and 12). Table 1 below shows ELISA plate-reader readouts for different time points from serum of patients and control as specified in FIG. 5.

TABLE 1

| 0 minutes | | | | | | | | | | | |
|---|---|---|---|---|---|---|---|---|---|---|---|
| 0.083 | 0.086 | 0.086 | 0.087 | 0.089 | 0.086 | 0.088 | 0.086 | 0.083 | 0.083 | 0.091 | 0.076 |
| 0.088 | 0.081 | 0.078 | 0.079 | 0.078 | 0.078 | 0.077 | 0.077 | 0.075 | 0.077 | 0.078 | 0.085 |
| 0.078 | 0.079 | 0.079 | 0.079 | 0.08 | 0.078 | 0.078 | 0.079 | 0.079 | 0.079 | 0.08 | 0.08 |
| 0.077 | 0.077 | 0.077 | 0.075 | 0.075 | 0.074 | 0.075 | 0.09 | 0.081 | 0.08 | 0.086 | 0.082 |
| 0.081 | 0.085 | 0.084 | 0.081 | 0.08 | 0.078 | 0.08 | 0.077 | 0.077 | 0.08 | 0.08 | 0.083 |
| 0.076 | 0.078 | 0.076 | 0.075 | 0.073 | 0.072 | 0.077 | 0.073 | 0.076 | 0.074 | 0.075 | 0.082 |
| 0.082 | 0.079 | 0.079 | 0.08 | 0.078 | 0.077 | 0.077 | 0.076 | 0.077 | 0.075 | 0.075 | 0.076 |
| 0.079 | 0.079 | 0.079 | 0.081 | 0.079 | 0.083 | 0.078 | 0.081 | 0.077 | 0.078 | 0.08 | 0.678 |
| 5 minutes | | | | | | | | | | | |
| 0.077 | 0.114 | 0.108 | 0.101 | 0.096 | 0.087 | 0.088 | 0.086 | 0.081 | 0.083 | 0.088 | 0.079 |
| 0.084 | 0.11 | 0.089 | 0.091 | 0.088 | 0.082 | 0.078 | 0.079 | 0.074 | 0.076 | 0.077 | 0.084 |
| 0.072 | 0.073 | 0.073 | 0.074 | 0.075 | 0.074 | 0.074 | 0.082 | 0.083 | 0.087 | 0.086 | 0.081 |
| 0.072 | 0.071 | 0.071 | 0.07 | 0.07 | 0.07 | 0.072 | 0.079 | 0.087 | 0.086 | 0.089 | 0.087 |
| 0.075 | 0.08 | 0.096 | 0.076 | 0.076 | 0.076 | 0.077 | 0.077 | 0.074 | 0.082 | 0.077 | 0.092 |
| 0.071 | 0.091 | 0.077 | 0.069 | 0.069 | 0.068 | 0.073 | 0.07 | 0.071 | 0.077 | 0.073 | 0.093 |
| 0.077 | 0.074 | 0.074 | 0.076 | 0.073 | 0.073 | 0.08 | 0.077 | 0.089 | 0.074 | 0.077 | 0.077 |
| 0.073 | 0.073 | 0.074 | 0.078 | 0.077 | 0.079 | 0.075 | 0.09 | 0.088 | 0.075 | 0.08 | *** |
| 10 minutes | | | | | | | | | | | |
| 0.076 | 0.136 | 0.125 | 0.115 | 0.11 | 0.094 | 0.095 | 0.091 | 0.086 | 0.083 | 0.089 | 0.083 |
| 0.084 | 0.136 | 0.104 | 0.11 | 0.105 | 0.093 | 0.087 | 0.088 | 0.081 | 0.08 | 0.085 | 0.092 |
| 0.069 | 0.071 | 0.071 | 0.073 | 0.074 | 0.072 | 0.074 | 0.091 | 0.095 | 0.103 | 0.103 | 0.093 |
| 0.07 | 0.071 | 0.072 | 0.07 | 0.07 | 0.07 | 0.073 | 0.092 | 0.1 | 0.1 | 0.1 | 0.104 |
| 0.073 | 0.078 | 0.111 | 0.075 | 0.076 | 0.077 | 0.079 | 0.079 | 0.075 | 0.089 | 0.079 | 0.113 |
| 0.068 | 0.09 | 0.079 | 0.069 | 0.07 | 0.069 | 0.073 | 0.073 | 0.072 | 0.087 | 0.078 | 0.123 |
| 0.072 | 0.069 | 0.072 | 0.076 | 0.071 | 0.072 | 0.083 | 0.082 | 0.104 | 0.076 | 0.084 | 0.086 |
| 0.07 | 0.072 | 0.073 | 0.077 | 0.075 | 0.079 | 0.075 | 0.101 | 0.103 | 0.076 | 0.087 | *** |
| 15 minutes | | | | | | | | | | | |
| 0.075 | 0.155 | 0.138 | 0.125 | 0.121 | 0.103 | 0.099 | 0.096 | 0.088 | 0.082 | 0.093 | 0.082 |
| 0.083 | 0.158 | 0.114 | 0.127 | 0.114 | 0.1 | 0.091 | 0.094 | 0.085 | 0.083 | 0.084 | 0.088 |
| 0.067 | 0.069 | 0.069 | 0.071 | 0.072 | 0.072 | 0.075 | 0.102 | 0.108 | 0.115 | 0.111 | 0.096 |
| 0.068 | 0.068 | 0.069 | 0.069 | 0.069 | 0.07 | 0.075 | 0.102 | 0.116 | 0.114 | 0.1 | 0.106 |
| 0.071 | 0.077 | 0.118 | 0.075 | 0.076 | 0.08 | 0.082 | 0.083 | 0.077 | 0.096 | 0.08 | 0.114 |
| 0.066 | 0.089 | 0.08 | 0.067 | 0.07 | 0.07 | 0.075 | 0.076 | 0.075 | 0.099 | 0.08 | 0.133 |
| 0.072 | 0.07 | 0.073 | 0.079 | 0.074 | 0.077 | 0.088 | 0.092 | 0.125 | 0.082 | 0.093 | 0.09 |
| 0.069 | 0.069 | 0.072 | 0.076 | 0.074 | 0.08 | 0.075 | 0.112 | 0.12 | 0.078 | 0.089 | *** |
| 30 minutes | | | | | | | | | | | |
| 0.071 | 0.182 | 0.158 | 0.14 | 0.127 | 0.103 | 0.098 | 0.093 | 0.086 | 0.084 | 0.094 | 0.085 |
| 0.08 | 0.192 | 0.122 | 0.131 | 0.121 | 0.101 | 0.088 | 0.091 | 0.082 | 0.085 | 0.089 | 0.094 |
| 0.064 | 0.065 | 0.066 | 0.067 | 0.069 | 0.068 | 0.071 | 0.103 | 0.114 | 0.123 | 0.123 | 0.109 |
| 0.063 | 0.063 | 0.065 | 0.065 | 0.065 | 0.066 | 0.072 | 0.1 | 0.12 | 0.125 | 0.111 | 0.117 |
| 0.066 | 0.072 | 0.125 | 0.071 | 0.073 | 0.079 | 0.08 | 0.082 | 0.074 | 0.098 | 0.079 | 0.142 |
| 0.062 | 0.087 | 0.077 | 0.064 | 0.065 | 0.066 | 0.074 | 0.073 | 0.071 | 0.102 | 0.082 | 0.169 |
| 0.066 | 0.064 | 0.069 | 0.075 | 0.068 | 0.072 | 0.093 | 0.095 | 0.142 | 0.08 | 0.094 | 0.099 |
| 0.064 | 0.064 | 0.067 | 0.073 | 0.07 | 0.077 | 0.072 | 0.116 | 0.139 | 0.075 | 0.093 | *** |
| 1 hour | | | | | | | | | | | |
| 0.072 | 0.255 | 0.209 | 0.178 | 0.156 | 0.12 | 0.111 | 0.105 | 0.096 | 0.096 | 0.106 | 0.098 |
| 0.084 | 0.278 | 0.154 | 0.169 | 0.154 | 0.121 | 0.105 | 0.108 | 0.096 | 0.1 | 0.106 | 0.114 |
| 0.062 | 0.063 | 0.065 | 0.066 | 0.069 | 0.068 | 0.075 | 0.124 | 0.141 | 0.157 | 0.16 | 0.137 |
| 0.065 | 0.065 | 0.066 | 0.066 | 0.067 | 0.069 | 0.08 | 0.121 | 0.153 | 0.164 | 0.138 | 0.151 |
| 0.066 | 0.073 | 0.132 | 0.071 | 0.076 | 0.083 | 0.087 | 0.09 | 0.079 | 0.113 | 0.083 | 0.191 |
| 0.063 | 0.088 | 0.078 | 0.063 | 0.067 | 0.068 | 0.08 | 0.08 | 0.076 | 0.122 | 0.093 | 0.243 |
| 0.065 | 0.062 | 0.067 | 0.076 | 0.069 | 0.075 | 0.108 | 0.109 | 0.181 | 0.087 | 0.113 | 0.127 |
| 0.063 | 0.064 | 0.067 | 0.075 | 0.072 | 0.081 | 0.076 | 0.14 | 0.172 | 0.081 | 0.111 | *** |
| 1 hr | | | | | | | | | | | |
| background | | | | | | | | | | | |
| subtracted | | | | | | | | | | | |
| 0.000 | 0.183 | 0.137 | 0.106 | 0.084 | 0.048 | 0.039 | 0.033 | 0.024 | 0.024 | 0.034 | 0.026 |
| 0.012 | 0.206 | 0.082 | 0.097 | 0.082 | 0.049 | 0.033 | 0.036 | 0.024 | 0.028 | 0.034 | 0.042 |
| −0.010 | −0.009 | −0.007 | −0.006 | −0.003 | −0.004 | 0.003 | 0.052 | 0.069 | 0.085 | 0.088 | 0.065 |
| −0.007 | −0.007 | −0.006 | −0.006 | −0.005 | −0.003 | 0.008 | 0.049 | 0.081 | 0.092 | 0.066 | 0.079 |
| −0.006 | 0.001 | 0.060 | −0.001 | 0.004 | 0.011 | 0.015 | 0.018 | 0.007 | 0.041 | 0.011 | 0.119 |
| −0.009 | 0.016 | 0.006 | −0.009 | −0.005 | −0.004 | 0.008 | 0.008 | 0.004 | 0.050 | 0.021 | 0.171 |
| −0.007 | −0.010 | −0.005 | 0.004 | −0.003 | 0.003 | 0.036 | 0.037 | 0.109 | 0.015 | 0.041 | 0.055 |
| −0.009 | −0.008 | −0.005 | 0.003 | 0.000 | 0.009 | 0.004 | 0.068 | 0.100 | 0.009 | 0.039 | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A composition for detecting the presence of cancer cells in a biological sample, the composition comprising an agent that specifically binds p62 or p90.

2. The composition of claim 1, wherein the cancer is an oral digestive cancer.

3. The composition of claim 1, wherein the agent that specifically binds p62 or p90 is an antibody.

4. A method of detecting cancer cells in a subject, the method comprising the steps of:
(a) providing a biological sample from the subject;
(b) detecting the amount of p62 or p90 in the sample;
(c) correlating the amount of the p62 or p90 in the sample with the presence of cancer in the subject.

5. The method of claim 4, wherein the cancer is an oral digestive cancer.

6. The method of claim 4, wherein the step (b) of detecting the amount of p62 or p90 in the sample comprises contacting the sample with at least one antibody that specifically binds the p62 or the p90.

7. The method of claim 4, wherein the step (b) of detecting the amount of p62 or p90 in the sample comprises performing an ELISA.

8. The method of claim 4 wherein the biological sample is a fluid selected from the group consisting of saliva, blood, and serum.

9. A kit for detecting oral or gastrointestinal cancer cells in a subject, the kit comprising:
(a) a solid substrate;
(b) at least one antibody that binds specifically to p90;
(c) an agent for detecting binding of the at least one antibody to the p90; and
(d) instructions for using the kit to detect oral or gastrointestinal cancer cells in a subject.

10. The kit of claim 9, wherein the at least one antibody is a monoclonal antibody.

11. The kit of claim 9, wherein the agent for detecting binding of the at least one antibody to P90 comprises a chromogenic substrate molecule.

12. The kit of claim 9, wherein detecting binding of the at least one antibody to P90 is correlated with cancer.
